(19) United States
(12) Patent Application Publication
(10) Pub. No.: US 2004/0097433 A1
(43) Pub. Date: May 20, 2004

(54) Chroman derivatives for the reduction of inflammation symptoms

(76) Inventors: Sekhar Boddupalli, San Jose, CA (US); Guy Miller, San Jose, CA (US); Bing Wang, Cupertino, CA (US)

Correspondence Address: Gloria Pfister, Galileo Pharmaceuticals, Inc., 5301 Patrick Henry Dr., Santa Clara, CA, 95054, US

(21) Appl. No.: 10/714,152

(22) Filed: Nov. 13, 2003

Related U.S. Application Data
(60) Provisional application No. 60/426,764, filed on Nov. 15, 2002.

(51) Int. Cl. A61K 31/7048; A61K 31/353

(52) U.S. Cl. 514/27; 514/454; 514/456

(57) ABSTRACT

The present invention provides a composition comprising chroman derivatives, for use in the reduction of inflammatory markers associated with inflammation, particularly for the reduction of C-reactive protein (CRP) and for use in the treatment and/or amelioration of symptoms of inflammation.

### CROSS-REFERENCE TO RELATED APPLICATIONS

This application claims benefit of U.S. provisional patent application serial No. 60/426,764, filed Nov. 15, 2002, which is incorporated by reference herein in its entirety.

### TECHNICAL FIELD

This invention generally relates to methods for treating and/or ameliorating the symptoms of inflammation in a mammalian subject with a composition comprising chroman derivatives.

### BACKGROUND

Inflammation is an important component of host protection, and is a composite response including successive events in response to an injury which may be infectious or non-infectious. Inflammation involves a variety of events on the cellular, molecular and physiologic levels. These events include vasodilatation; increased vascular permeability; extravasation of plasma leading to interstitial edema; chemotaxis of neutrophils, macrophages and lymphocytes; cytokine production; acute phase reactants; leukocytosis; fever; increased metabolic rate; impaired albumin production and hypoalbuminemia; activation of complement; and stimulation of antibodies. Inflammation is associated with diseases or disorders such as, for example, neurodegenerative diseases, SIRS, asthma, diabetes associated nephropathy and retinopathy, protein wasting, muscle fatigue or inflammation and PMS, infectious diseases, as well as various cardiovascular disorders.

Biochemical markers of inflammation are known in the art and include C-reactive protein (CRP) and members of the interleukin family. The presence of elevated levels of certain of these markers has been shown to be associated with development of disease. For example, CRP has been reported as a marker for systemic inflammation Spanheimer (2001, Postgrad. Med. 109(4) 26) and Ridkler et al. (2000, N.E.J.M. 342(12):836-43).

U.S. Pat. Nos. 6,410,589; 6,242,479; 6,150,402; 6,083,982; 6,048,891; and US Patent Application No. 2001/0031782 disclose gamma-tocopherol metabolites and chroman derivatives of the present invention, methods for isolating and synthesizing the compounds and methods of use as natriuretic and antioxidant compounds, but these applications neither teach nor suggest their use for the reduction of biochemical markers associated with inflammation, particularly the use for the reduction of CRP. Reduction of the inflammation marker CRP with gamma tocopherol has been disclosed in commonly owned US patent applications 2003/0100603 and 2003/0144219.

The disclosure of all patents and publications cited herein are incorporated by reference in their entirety.

There remains a need for compositions and methods for reducing one or more biochemical markers of inflammation, thereby reducing or ameliorating the symptoms of inflammation associated with disease. Particularly there remains a need for methods for reducing elevated CRP levels associated with a number of diseases and disorders including, but not limited to, inflammation, cardiovascular inflammatory disease, diabetes and infectious diseases.

### DISCLOSURE OF THE INVENTION

The present invention relates to methods for treating and/or ameliorating the symptoms of inflammation in a mammalian subject. The present invention provides methods for reducing the levels of inflammation markers and proteins associated with inflammation, such as for example CRP; cytokines associated with inflammation, including IL-1 through 17, TNF-alpha and B61. The present invention provides methods of reducing levels of C-reactive protein (CRP) associated inflammation symptoms in a mammalian subject, comprising administering to the subject a composition comprising a chroman derivative in a pharmaceutically effective amount, and by said administering, reducing symptoms associated with said inflammation.

In a preferred embodiment the compositions comprise a compound of Formula I:

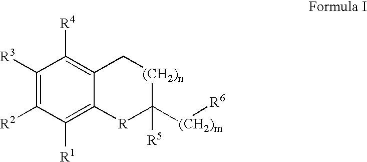

wherein:

R is O, S, SO, SO₂, a secondary or tertiary amine, a phosphate, a phosphoester, or an unsubstituted or substituted methylene group;

R¹ and R² independently are H, OH, alkyl, aryl, alkenyl, alkynyl, ether, ester, amine, amide, halogen, or sulfonyl, or jointly complete a 5- or 6-membered aliphatic or aromatic ring;

R³ and R⁴ independently are H, OH, alkyl, aryl, alkenyl, alkynyl, ether, ester, amine, amide, nitro, halogen, or sulfonyl, or jointly complete a 5- or 6-membered aliphatic, aromatic, or heterocyclic ring;

R⁵ is H, OH, alkyl, aryl, alkenyl, alkynyl, ester, or amine;

R⁶ is COOH, COOR⁷, CONH₂, CONHR⁷, CONR⁷R⁸, NH₂, NHR⁷, NR⁷R⁸, OH, or OR⁹,

R⁷ and R⁸ independently are alkyl, aryl, aralkyl, alkenyl, or alkynyl;

R⁹ is alkyl, aralkyl, alkenyl, alkynyl, or a glucoside;

n is 0 to 3; and

m is 0 to 5;

or individual isomer, racemic or non racemic mixture of isomers, of pharmaceutically acceptable salt or solvate thereof.

In preferred embodiments, the compounds are selected from the group

6-Hydroxy-2,5,7,8-tetramethyl-chroman-2-carboxylic acid;

6-Hydroxy-2,5,7,8-tetramethyl-chroman-2-carboxylic acid (adamantan-2-ylmethyl)-amide;

2-Hydroxymethyl-6-(6-hydroxy-2,5,7,8-tetramethyl-chroman-2-ylmethoxy)-tetrahydro-pyran-3,4,5-triol;

3-(6-Hydroxy-2-methyl-chroman-2-yl)-propionic acid methyl ester;

3-(6-Hydroxy-2,7,8-trimethyl-chroman-2-yl)-propionic acid methyl ester;

3-(6-Hydroxy-2,7,8-trimethyl-chroman-2-yl)-propionic acid;

3-[8-(2-Methoxycarbonyl-ethyl)-3,5,6,8-tetramethyl-1,2,3,8,9,10-hexahydro-pyrano[3,2-f]chromen-3-yl]-propionic acid methyl ester;

3-[8-(2-Carboxy-ethyl)-3,5,6,8-tetramethyl-1,2,3,8,9,10-hexahydro-pyrano[3,2-f]chromen-3-yl]propionic acid;

3-(6-Hydroxy-2-methyl-chroman-2-yl)-propionic acid;

3-(6-Hydroxy-2,5,7,8-tetramethyl-chroman-2-yl)-propionic acid;

3-(2,5,7,8-Tetramethyl-chroman-2-yl)-propionic acid;

3-(6-Hydroxy-2,7,8-trimethyl-5-nitro-chroman-2-yl)-propionic acid;

3-(6-Hydroxy-2-methyl-3,4-dihydro-2H-benzo[h]chromen-2-yl)-propionic acid;

3-(5-Bromo-6-hydroxy-2,7,8-trimethyl-chroman-2-yl)-propionic acid methyl ester;

3-(7,8-Dihydroxy-2-methyl-chroman-2-yl)-propionic acid; and

6-Hydroxy-2,5,7,8-tetramethyl-chroman-2-carboxylic acid.

In another embodiment the chroman derivative is a metabolite of tocopherol.

In another embodiment the compounds are selected from 3-(6-hydroxy-2,7,8-trimethyl-chroman-2-yl)propionic acid methyl ester and 3-(6-hydroxy-2,7,8-trimethyl-chroman-2-yl)-propionic acid. In another embodiment the compounds are selected from 3-(5-bromo-6-hydroxy-2,7,8-trimethyl-chroman-2-yl)propionic acid methyl ester and 3-(5-bromo-6-hydroxy-2,7,8-trimethyl-chroman-2-yl)-propionic acid.

In another embodiment the chroman derivative is a metabolite of tocopherol.

The present invention provides methods of reducing the level of C-reactive protein (CRP) in an individual subject to a CRP associated inflammatory condition comprising administering to the individual an effective amount of a composition comprising a chroman derivative. The present invention provides methods of reducing the level of cytokines in an individual subject to an inflammatory condition comprising administering to the individual an effective amount of a composition comprising a chroman derivative.

In some embodiments the invention provides methods of reducing inflammation in an individual subject to an inflammation condition comprising cardiovascular diseases or disorders, including atrial fibrillation, unstable angina, coronary artery disease, peripheral artery disease, cardiac allograft vasculopathy (CAVD); mastitis; preclampsia; inflammatory bowel conditions; stroke; tissue infarction; lumbosciatic; estrogen/progestin hormone replacement therapy (HRT); infection (bacterial, viral and protozoan); bacterial meningitis; trauma; surgery; biomaterial implants; smoking; obesity; neurodegenerative diseases such as, Alzheimer's; infectious disease, such as, for example, myocarditis, cardiomyopathy, acute endocarditis, pericarditis; atherosclerosis; Systemic Inflammatory Response Syndrome (SIRS)/sepsis; adult respiratory distress syndrome (ARDS); asthma; rheumatoid arthritis, osteoarthritis, systemic lupus erythematosis; Airway hyper-responsiveness (AHR); bronchial hyper-reactivity; Chronic Obstructive Pulmonary disease (COPD); Congestive Heart Failure (CHF); inflammatory complications of diabetes mellitus type I and type II; metabolic syndrome; end stage renal disease (ESRD), pre-menstrual syndrome (PMS), muscle fatigue or inflammation; multiple organ dysfunction syndrome (MODS); airway hyper-responsiveness (AHR); bronchial hyper-reactivity; aging; acute allergic reactions; gingivitis and dermal conditions. In a preferred embodiment the invention provides methods of reducing the elevated level of a marker associated with inflammation. In a preferred embodiment the marker is CRP.

### BEST MODE FOR CARRYING OUT THE INVENTION

The present invention provides methods for reducing inflammation with a composition comprising a chroman derivative. In some examples, the present invention provides methods for reducing elevated levels of CRP, thereby ameliorating an inflammatory symptom associated with disease or an inflammatory condition and/or reducing a mammalian subject's risk of progressing into long term or chronic inflammatory conditions. In some examples, the present invention provides methods for maintaining normal or healthy levels of inflammatory markers in subjects. In other examples, the present invention provides methods for maintaining normal or healthy levels of CRP in subjects.

Inflammation is associated with for example, neurodegenerative diseases such as, Alzheimer's; cardiovascular diseases or disorders; infectious disease, such as, for example, myocarditis, cardiomyopathy, acute endocarditis, pericarditis; atherosclerosis; Systemic Inflammatory Response Syndrome (SIRS)/sepsis; adult respiratory distress syndrome (ARDS); asthma; rheumatoid arthritis, osteoarthritis, systemic lupus erythematosis; Airway hyperresponsiveness (AHR); bronchial hyperreactivity; Chronic Obstructive Pulmonary disease (COPD); Congestive Heart Failure (CHF); inflammatory complications of diabetes mellitus; end stage renal disease (ESRD), pre-menstrual syndrome (PMS), muscle fatigue or inflammation; and dermal conditions.

The present invention provides compositions comprising chroman compositions, including but not limited to compositions comprising certain tocopherol metabolites, and methods for reducing the level of the inflammatory markers associated with inflammation, such as for example, reducing pain, and/or reducing edema and/or reducing fatigue associated with inflammation.

In other examples, the present invention provides methods for maintaining healthy or normal levels of C-Reactive Protein (CRP) in a mammalian subject at risk for inflammation or inflammatory condition(s) associated with the diseases or disorders disclosed herein, comprising administering to the subject a composition comprising a chroman derivative, including a composition comprising certain tocopherol metabolites, in a pharmaceutically effective amount, and by said administering, maintaining healthy or normal levels of CRP in said mammalian subject.

Definitions

Inflammation is associated with diseases, disorders and conditions such as for example, cardiovascular diseases or disorders; neurodegenerative diseases such as, Alzheimer's; infectious diseases, such as, for example, myocarditis, cardiomyopathy, acute endocarditis, pericarditis; atherosclerosis; Systemic Inflammatory Response Syndrome (SIRS)/sepsis; adult respiratory distress syndrome (ARDS); asthma; rheumatoid arthritis, osteoarthritis, systemic lupus erythematosis; Airway hyperresponsiveness (AHR); bronchial hyperreactivity; Chronic Obstructive Pulmonary disease (COPD); Congestive Heart Failure (CHF); inflammatory complications of diabetes mellitus; ESRD; pre-menstrual syndrome (PMS); muscle fatigue or inflammation, and dermal conditions. As used herein, “respiratory inflammatory conditions” refer to SIRS, ARDS, asthma and AHR.

Elevated levels of C-reactive protein (CRP) have been associated with various inflammatory conditions. As used herein, “CRP associated inflammation” refers to inflammatory conditions and/or inflammation associated with elevated levels of CRP such as for example, cardiovascular diseases or disorders, including atrial fibrillation, unstable angina, coronary artery disease, peripheral artery disease, cardiac allograft vasculopathy (CAVD); mastitis; preclampsia; inflammatory bowel conditions; stroke; tissue infarction; lumbosciatic; estrogen/progestin hormone replacement therapy (HRT); infection (bacterial, viral and protozoan); bacterial meningitis; trauma; surgery; biomaterial implants; smoking; obesity; neurodegenerative diseases such as, Alzheimer's; infectious disease, such as, for example, myocarditis, cardiomyopathy, acute endocarditis, pericarditis; atherosclerosis; Systemic Inflammatory Response Syndrome (SIRS)/sepsis; adult respiratory distress syndrome (ARDS); asthma; rheumatoid arthritis, osteoarthritis, systemic lupus erythematosis; Airway hyper-responsiveness (AHR); bronchial hyper-reactivity; Chronic Obstructive Pulmonary disease (COPD); Congestive Heart Failure (CHF); inflammatory complications of diabetes mellitus type I and type II; metabolic syndrome; end stage renal disease (ESRD), pre-menstrual syndrome (PMS) or muscle fatigue or inflammation; multiple organ dysfunction syndrome (MODS); airway hyper-responsiveness (AHR); bronchial hyper-reactivity; aging; acute allergic reactions; gingivitis and dermal conditions.

“Tocopherol” refers to alpha-, beta-, gamma-, or delta-tocopherol, preferably gamma-tocopherol.

As used herein, a “tocopherol metabolite” refers to a metabolite of a tocopherol. See for example, Wechter et al., U.S. Pat. Nos. 6,242,479, 6,410,589; 6,242,479, 6,150,402, 6,083,982, 6,048,891, and US 2001/0031782, hereby incorporated by reference in its entirety, for the description of gamma-tocopherol metabolites.

An “effective amount” is an amount sufficient to effect beneficial or desired results. An effective amount can be administered in one or more administrations. For purposes of this invention, an effective amount of a chroman composition is an amount that is sufficient to reduce CRP. For example, in Example 1A disclosed herein, 3-(6-hydroxy-2,7,8-trimethyl-chroman-2-yl)-propionic acid methyl ester and 3-(6-hydroxy-2,7,8-trimethyl-chroman-2-yl)-propionic acid were shown to be effective at reducing CRP levels. Amelioration is at least about 30% reduction in the levels of CRP.

A “mammalian subject” or “individual” (used interchangeably herein) includes, but is not limited to, a human, a farm animal, a sport animal, and a pet.

By “amelioration” is meant the prevention, reduction or palliation of a state, or improvement of the state of a subject; the amelioration of a stress is the counter-acting of the negative aspects of a stress. Amelioration includes, but does not require complete recovery or complete prevention of a stress.

By “treatment” or “treating” is meant any treatment of a disease or disorder, in a mammal, including: preventing or protecting against the disease or disorder, that is, causing, the clinical symptoms of the disease not to develop; inhibiting the disease, that is, arresting or suppressing the development of clinical symptoms; and/or relieving the disease, that is, causing the regression of clinical symptoms.

The term “alkenyl” refers to a monoradical branched or unbranched, unsaturated or polyunsaturated hydrocarbon chain, having from about 2 to 20 carbon atoms, more preferably about 2 to 10 carbon atoms. The term alkenyl includes both “unsubstituted alkenyls” and “substituted alkenyls”, the latter of which refers to alkenyl moieties having substituents replacing a hydrogen on one or more carbons of the hydrocarbon backbone. Such substituents can include, but are not limited to hydroxy, alkoxy, alkyl, alkenyl, nitro, carboxy, carbonyl, amino, or halogen. This term is exemplified by groups such as ethenyl, but-2-enyl, e-methyl-but-2-enyl (also referred to as “prenyl”, octa-2,6-dienyl, 3,7-dimethyl-octa-2,6-dienyl (also referred to as “geranyl”), and the like.

The term “alkyl” refers to a monoradical branched or unbranched saturated hydrocarbon chain preferably having from about 1 to 20 carbon atoms, more preferably about 1 to 10 carbon atoms, and even more preferably about 1 to 6 carbon atoms. This term is exemplified by groups such as methyl, ethyl, n-propyl, iso-propyl, n-butyl, iso-butyl, n-hexyl, n-decyl, tetradecyl, and the like. The term “lower alkyl refers to a monoradical branched or unbranched saturated hydrocarbon chain of 1 to 6 atoms. The term alkyl includes both “unsubstituted alkyls” and “substituted alkyls”, the latter of which refers to alkyl moieties having substituents replacing a hydrogen on one or more carbons of the hydrocarbon backbone. Such substituents can include, but are not limited to hydroxy, alkoxy, alkyl, alkenyl, nitro, carboxy, carbonyl, amino, or halogen.

The term “alkylene” refers to a diradical derived from the above-defined monoradical, alkyl. This term is exemplified by groups such as methylene (—CH₂—), ethylene (—CH₂CH₂—), the propylene isomers [e.g., —CH₂CH₂CH₂— and —CH(CH₃)CH₂—] and the like.

The term “alkynyl” refers to a monoradical branched or unbranched, unsaturated or polyunsaturated hydrocarbon chain, having from about 2 to 20 carbon atoms, more preferably about 2 to 10 carbon atoms, containing at list one triple bond. The term alkynyl includes both “unsubstituted alkynyls” and “substituted alkynyls”, the latter of which refers to alkynyl moieties having substituents replacing a hydrogen on one or more carbons of the hydrocarbon backbone. Such substituents can include, but are not limited to hydroxy, alkoxy, alkyl, alkenyl, nitro, carboxy, carbonyl, amino, or halogen. This term is exemplified by groups such as ethynyl, propynyl, butynyl, pentynyl, hexynyl, heptynyl, octynyl, nonynyl, decynyl, and the like.

The term “amino” refers to the group —NH₂ as well as to the groups —NHR or —NRR where each R is independently selected from the group: optionally substituted alkyl, optionally substituted cycloalkyl, optionally substituted alkenyl, optionally substituted cycloalkenyl, optionally substituted alkynyl, optionally substituted aryl, optionally substituted heterocyclyl, acyl, optionally substituted alkoxy, carboxy and alkoxycarbonyl, and where —NRR may be a cyclic amine.

The term “aralkyl” refers to the moiety “-alkylene-aryl”, each having the meaning as defined herein.

The term “aryl” refers to an aromatic cyclic hydrocarbon group of from 6 to 20 carbon atoms having a single ring (e.g., phenyl) or multiple condensed (fused) rings (e.g., naphthyl or anthryl). Preferred aryls include phenyl, naphthyl and the like. The term aryl includes both “unsubstituted aryls” and “substituted aryls”, the latter of which refers to aryl moieties having substituents replacing a hydrogen on one or more carbons of the ring. Such substituents can include, but are not limited to hydroxy, alkoxy, alkyl, alkenyl, nitro, carboxy, carbonyl, amino, or halogen.

The term “halo” or “halogen” refers to fluoro, chloro, bromo, and iodo.

The terms “heterocycle”, “heterocyclic” and “heterocyclyl” refer to a monoradical, saturated, partially unsaturated or unsaturated (aromatic), cyclic hydrocarbon group having about 1 to 40 (preferably from about 3 to 15) carbon atoms and about 1 to 10 hetero atoms (preferably about 1 to 4 heteroatoms, selected from nitrogen, sulfur, phosphorus, and/or oxygen) within the ring. Such heterocyclic groups can have a single ring or multiple condensed rings. The term heterocycle includes both “unsubstituted heterocycles” and “substituted heterocycles”, the latter of which refers to heterocycle moieties having substituents replacing a hydrogen on one or more carbons of the ring. Such substituents can include, but are not limited to hydroxy, alkoxy, alkyl, alkenyl, nitro, carboxy, carbonyl, amino, or halogen. Preferred heterocycles include morpholino, piperidinyl, thiazolidine, pyrazol, tetrahydropyranyl, and the like.

The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

The term “pharmaceutically acceptable salt” refers to salts which retain the biological effectiveness and properties of the compounds of this invention and which are not biologically or otherwise undesirable. In many cases, the compounds of this invention are capable of forming acid and/or base salts by virtue of the presence of amino and/or carboxyl groups or groups similar thereto. Pharmaceutically acceptable base addition salts can be prepared from inorganic and organic bases. Salts derived from inorganic bases, include by way of example only, sodium, potassium, lithium, ammonium, calcium and magnesium salts. Salts derived from organic bases include, but are not limited to, salts of primary, secondary and tertiary amines, such as alkyl amines, dialkyl amines, trialkyl amines, substituted alkyl amines, di(substituted alkyl) amines, tri(substituted alkyl) amines, alkenyl amines, dialkenyl amines, trialkenyl amines, substituted alkenyl amines, di(substituted alkenyl) amines, tri(substituted alkenyl) amines, cycloalkyl amines, di(cycloalkyl) amines, tri(cycloalkyl) amines, substituted cycloalkyl amines, disubstituted cycloalkyl amine, trisubstituted cycloalkyl amines, cycloalkenyl amines, di(cycloalkenyl) amines, tri(cycloalkenyl) amines, substituted cycloalkenyl amines, disubstituted cycloalkenyl amine, trisubstituted cycloalkenyl amines, aryl amines, diaryl amines, triaryl amines, heterocyclic amines, diheterocyclic amines, triheterocyclic amines, mixed di- and tri-amines where at least two of the substituents on the amine are different and are selected from the group consisting of alkyl, substituted alkyl, alkenyl, substituted alkenyl, cycloalkyl, substituted cycloalkyl, cycloalkenyl, substituted cycloalkenyl, aryl, heterocyclic, and the like. Also included are amines where the two or three substituents, together with the amino nitrogen, form a heterocyclic group.

Specific examples of suitable amines include, by way of example only, isopropylamine, trimethyl amine, diethyl amine, tri(iso-propyl) amine, tri(n-propyl) amine, ethanolamine, 2-dimethylaminoethanol, tromethamine, lysine, arginine, histidine, caffeine, procaine, hydrabamine, choline, betaine, ethylenediamine, glucosamine, N-alkylglucamines, theobromine, purines, piperazine, piperidine, morpholine, N-ethylpiperidine, and the like.

Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like.

The term “sulfonyl” refers to the groups: —S(O₂)-(optionally substituted alkyl), —S(O₂)-optionally substituted aryl), —S(O₂)-(optionally substituted heterocyclyl).

General Methods

General techniques for chemical manipulations are known in the art and are generally described in, for example, Some Modern Methods of Organic Synthesis, Third Edition, Cambridge University Press; and Warren (1978) Designing Organic Syntheses, John Wiley & Sons, Ltd. Molecular biology techniques are generally described in, for example, Sambrook et al. (1989), Molecular Cloning: A Laboratory Manual, Second Edition; and Ausubel et al., eds. (1987) Current Protocols In Molecular Biology. Reagents useful in applying these techniques are widely known in the art and commercially available from a number of vendors.

Inflammatory Markers

A number of proximal mediators of the inflammatory response have been identified and include the inflammatory cytokines, interleukin-1β (IL-1β) (U.S. Pat. No. 6,210,877) and tumor necrosis factor alpha (TNF-α), as described in U.S. Pat. Nos. 5,993,811 6,210,877 and 6,203,997. Other molecules have been reported for use as markers of systemic inflammation, including for example, CRP (Ridker et al. 2000 N.E.J.M. 342(12):836-43; Spanheimer supra); certain cellular adhesion molecules such as sICAM-1 (U.S. Pat. No. 6,049,147); and B61 (U.S. Pat. No. 5,688,656). Other proteins associated with inflammation include leukotriene, thromboxane, and isoprostane. Other proteins or markers associated with inflammation include serum amyloid A protein, fibrinectin, fibrinogen, leptin, prostaglandin E2, serum procalcitonin, soluble TNF receptor 2, and elevated white blood count, including percent and total granulocytes (polymorphonuclear leukocytes) monocytes, lymphocytes and eosinophils.

C-reactive protein (CRP) is an acute phase protein in humans that increases rapidly in concentration as a result of systemic inflammation, for example, as a result of tissue injury, inflammation or infection and in IDDM patients without macrovascular disease. Generally speaking, the normal range of CRP in human serum is 0.08-2 milligram (mg) per liter. CRP levels can increase between 100-1000-fold during an inflammatory response. Elevated serum levels of CRP are seen 6-12 hours after an inflammatory stimulus, and maximum levels are reached within 48-72 hours. Generally, CRP levels will return to normal 55-10 days after remission of inflammation. Because the accumulation of CRP in serum closely parallels the course of inflammation and tissue injury, CRP has been used as a diagnostic tool to detect inflammation and to monitor the clinical course of a number of diseases. For example, CRP levels are found to exceed 50 mg/l in rheumatoid arthritis, systemic lupus erythematosus (SLE), ulcerative colitis, Crohn's disease, acute pancreatitis, cardiac infarction, septicemia, bacterial meningitis, and pneumonia. Further, CRP levels have been correlated with increased risk of cardiovascular disease and stroke (Lagrand, W. K., et al, Circulation 100: 96-102, 1999). CRP levels are also elevated during inflammatory disorders such as infection, trauma, surgery, tissue infarction, and in IDDM patients without macrovascular disease. The magnitude of the increase varies from about 50% to as much as 100-fold during systemic inflammation (Gabay, C., et al., New Engl. J. Med. 340: 448-454, 1999). Most CRP is produced in hepatocytes in response to pro-inflammatory cytokines, especially interleukin-6 and 1β (Ganter, U., et al., EMBO J. 8: 3773-3779, 1989), although macrophages have also been reported to release CRP (Dong, Q, et al, J. Immunol. 156: 481504820, 1996).

Elevated CRP levels have been reported in a number of inflammatory conditions, including, but not limited to insulin-dependent diabetes mellitus (IDDM; Type I; (Schalkwijk, C G., et al., 1999, Diabetologia 42(3): 351-7), non-insulin-dependent diabetes mellitus (NIDDM; Type II), metabolic syndrome, cardiovascular disease, atrial fibrillation (Chung M. K, et al, 2001, Circulation 104(24): 2886-91), paroxysmal atrial fibrillation (Dernellis, J., et al., 2001, Acta Cardiologica 56(6): 375-80), cardiac allograft vasculopathy (CAVD; in heart transplant patients) Pethig, K., et al., 2000, Circulation 102: 111233-6), mastitis (WO 9522767), pre-eclampsia, peripheral artery disease, inflammatory bowl disorders (e.g., Crohn's disease; Poullis, A. P., et al., Eur J. Gastro Hepat 14(4):409-412 (2002), stroke, tissue infarction, Lumbosciatic syndrome (local nerve root impingement) (Le Gars, L, et al., 2001, Bone, Joint, Spine: Revue du Rhumatisme 67(5): 452-5), uremic patients having end-stage renal disease (ESRD), or inflammation-associated conditions such as infection (bacterial, viral, and protozoal), bacterial meningitis (Shimetani, N, et al., 2001, Scan. J. Clin Lab. Invest. 61(7): 567-74), trauma, surgery, sepsis (Tschaikowsky, K., et al., 2002, Critical Care Med. 30(5): 1015-23), biomaterial implants (Lobler, M., et al., J. Biomaterials Research 61(1): 165-167), smoking, obesity, premenstrual syndrome, rheumatoid arthritis, aging. Women taking hormone replacement therapy (estrogen+progestin; HRT) were also found to have elevated CRT and increased risk of cardiovascular events (Ridker, P M, et al, 1999, Circulation 100: 713-716; Hulley, S., et al., 1998, JAMA 280: 605-613).

Other inflammatory conditions associated with increased CRP levels include acute allergic reactions (Lin, R. Y., et al, 2001, Ann Allergy Asth Immunol 87(5): 412-16), respiratory conditions, such as asthma (Yamaguchi, A., et al., 2000, J. Clin. Pharmacol. 40(3): 284-9), COPD (Malo, 0, et al., 2002, Arch Bronconeumol 38(4):172-6), or the like, periodontal disease, such as gingivitis (Glurich, I., et al., 2002, Clin. Diag. Lab. Immunol. 9(2): 425-32; Noack, B., et al., 2001, J. Periodontology, 72(9): 1221-7).

Recent studies have shown that mortality in patients having coronary artery disease can be correlated with high levels of CRP (Bickel, C., et al., 2002, Am. J. Cardiology 89(8):901-908, 2002; Jialal, I. and Devaraj, S. Am. J. Clin Path 116 Suppl: S108-15, 2001). In a large prospective study, patients with unstable angina and elevated C-reactive protein levels had a 3-fold higher risk of coronary events during a 90-day follow-up. (Ferreiros, et al, 1999, Circulation 99: 237-42). Similarly, elevated levels of IL-1 and IL-6 have been shown to be associated with aneurysm associated with unstable angina (Biasucci, L. M., 1999, Circulation 99: 2079-2084). Further, elevated CRP levels were associated with a doubling of risk of ischemic stroke in hypertensive patients (DiNapoli, M., et al., 2001, Stroke 32: 133-138) and with increased risk of developing age-related cataracts (Schaumberg, D. A., et al., 1999, Ann. Epidemiol 9: 166-171).

Likewise, in uremic patients (having ESRD), elevated CRP levels may contribute to enhanced CV morbidity and mortality. Elevated CRP levels are also associated with Type II diabetes, obesity and may also be predictive of mortality in these conditions (Festa, A, et al., 2003, Circulation 108, 1822-1830).

In some examples, the present invention provides compositions comprising chroman derivatives including, but not limited to compositions comprising tocopherol metabolites for use in reducing inflammation, in particular for use in reducing CRP associated with inflammation and/or inflammatory conditions including cardiovascular diseases or disorders, such as atrial fibrillation, unstable angina, coronary artery disease, peripheral artery disease, cardiac allograft vasculopathy (CAVD); mastitis; preclampsia; inflammatory bowel conditions; stroke; tissue infarction; lumbosciatic; estrogen/progestin hormone replacement therapy (HRT); infection (bacterial, viral and protozoan); bacterial meningitis; trauma; surgery; biomaterial implants; smoking; obesity; neurodegenerative diseases such as, Alzheimer's; infectious disease, such as, for example, myocarditis, cardiomyopathy, acute endocarditis, pericarditis; atherosclerosis; Systemic Inflammatory Response Syndrome (SIRS)/sepsis; adult respiratory distress syndrome (ARDS); asthma; rheumatoid arthritis, osteoarthritis, systemic lupus erythematosis; Airway hyper-responsiveness (AHR); bronchial hyper-reactivity; Chronic Obstructive Pulmonary disease (COPD); Congestive Heart Failure (CHF); inflammatory complications of diabetes mellitus type I and type II; metabolic syndrome; end stage renal disease (ESRD), pre-menstrual syndrome (PMS) or muscle fatigue or inflammation; multiple organ dysfunction syndrome (MODS); airway hyper-responsiveness (AHR); bronchial hyper-reactivity; aging; acute allergic reactions; periodontal disease, such as gingivitis, and dermal conditions including inflammatory skin conditions.

In some examples of the present invention, a 3-(6-hydroxy-2,7,8-trimethyl-chroman-2-yl)-propionic acid methyl ester enriched composition is used in methods for the treatment and/or amelioration of a symptom of inflammation, such as in methods for reducing CRP levels associated with the inflammation, including respiratory inflammatory conditions, such as SIRS, ARDS, AHR, and asthma; sepsis; diabetes; muscle fatigue; systemic lupus erythematosis (SLE); renal inflammation, including in ESRD; PMS, and periodontal disease.

In some examples of the present invention, a 3-(6-hydroxy-2,7,8-trimethyl-chroman-2-yl)-propionic acid composition is used in methods for the treatment and/or amelioration of a symptom of inflammation, such as in methods for reducing CRP levels associated with the inflammation, including cardiovascular inflammation, respiratory inflammatory conditions, such as SIRS, ARDS, AHR, and asthma; sepsis; diabetes; muscle fatigue; systemic lupus erythematosis (SLE); renal inflammation, including in ESRD; PMS, periodontal disease and inflammatory skin conditions.

In some examples of the present invention, a 3-(5-bromo-6-hydroxy-2,7,8-trimethyl-chroman-2-yl)-propionic acid methyl ester composition is used in methods for the treatment and/or amelioration of a symptom of inflammation, such as in methods for reducing CRP levels associated with the inflammation, including cardiovascular inflammation, respiratory inflammatory conditions, such as SIRS, ARDS, AHR, and asthma; sepsis; diabetes; muscle fatigue; systemic lupus erythematosis (SLE); renal inflammation, including in ESRD; PMS, periodontal disease and inflammatory skin conditions.

In some examples of the present invention, a 3-(5-bromo-6-hydroxy-2,7,8-trimethyl-chroman-2-yl)-propionic acid composition is used in methods for the treatment and/or amelioration of a symptom of inflammation, such as in methods for reducing CRP levels associated with the inflammation, including cardiovascular inflammation, respiratory inflammatory conditions, such as SIRS, ARDS, AHR, and asthma; sepsis; diabetes; muscle fatigue; systemic lupus erythematosis (SLE); renal inflammation, including in ESRD; PMS, periodontal disease and inflammatory skin conditions.

Activity of chroman derivatives can be experimentally tested, for example, in an assay which measures the level of the CRP inflammatory marker or in the E-selectin assay, also known as the Endothelial-Leukocyte Adhesion Molecule (ELAM) assay, associated with inflammation. Such assays are detailed in Example 1 and are known to those of skill in the art. Compounds of the present invention have also shown activity in neuronal protection of hippocampal cells as detailed in the Hippocampal Anoxia-Reoxigenation Cell Death. Such assay is described Example 2.

Specific Markers and Assays for Inflammation

A number of proximal mediators of the inflammatory response have been identified and include the inflammatory cytokines, interleukin-1β (IL-1β) (U.S. Pat. No. 6,210,877) and tumor necrosis factor alpha (TNF-α), as described in U.S. Pat. Nos. 5,993,811 6,210,877 and 6,203,997. Other molecules have been reported for use as markers of systemic inflammation, including for example, CRP (Ridker et al. 2000 N.E.J.M. 342(12):836-43; Spanheimer supra); certain cellular adhesion molecules such as sICAM-1 (U.S. Pat. No. 6,049,147); and B61 (U.S. Pat. No. 5,688,656). Other proteins associated with inflammation include leukotriene, thromboxane, and isoprostane.

There presently are commercial sources which produce reagents for assays for C-reactive protein, for example, but not limited to, CalBiochem (San Diego, Calif.). B61 is secreted by endothelial cells, fibroblasts and keratinocytes in response to lipopolysaccharide and the pro-inflammatory cytokines IL-1 and TNF. The B61 gene product is not, however, induced in response to other agents such as growth factors and interferon, thus induction of B61 is thus highly specific to inflammation (U.S. Pat. No. 5,688,656). The presence of B61 transcript can be detected directly by in situ hybridization using probes of encoding cDNA. Alternatively, the B61 protein can be measured in biological fluids such as plasma, cerebrospinal fluid or urine using an antibody-based assay. These assay procedures known in the art and described in particular in U.S. Pat. No. 5,688,656, are useful in both prognostic and diagnostic applications.

In studies carried out in support of the present invention, a combination of Interleukin-1β, IL-6, and dexamethasone is used to induce CRP production, and counter-agents are tested for their ability to reduce this production in cultured liver cells, as detailed in Example 1A. The assay is performed on cells grown in 96-well format allowing high throughput screening of compounds. As described herein, chroman derivatives were able to reduce CRP levels in an assay such as the one described in Example 1A.

Another useful cell screening assay, exemplified herein in Example 1B, is the E-selectin (ELAM) production assay, which measures activity of test compounds in reducing expression of ELAM in activated endothelial cells. Briefly, endothelial cells are activated by adding known activators such as lipopolysaccharide, TNF-α, or IL-1β, alone or in some combination. Activated cells produce ELAM, which can be measured using, for example, an E-selectin monoclonal antibody-based ELISA assay. In studies carried out in support of the present invention, ELAM production was decreased. As described herein, chroman derivatives were able to reduce ELAM production in an assay such as the one described in Example 1B.

In another experiment carried out in support of the present invention according to methods detailed in the Examples, anoxia was induced in primary cultures of hippocampal neuronal cells, and compounds were tested for their ability to prevent cell death. Primary cultures of hippocampal neurons are used to test compounds for activity in neuronal protection. Hippocampal cultures are typically prepared from 18- to 19-day fetal rats. At this age the generation of the pyramidal neurons, which begins in the rat at about E 15, is essentially complete. The brain tissue at this stage is relatively easy to dissociate, the meninges are removed readily, and the number of glial cells still is relatively modest (Park L C et al., Metabolic impairment elicits brain cell type-selective changes in oxidative stress and cell death in culture. Neurochem. 74 (1): 114-124).

In vivo evaluation of anti-inflammatory activity can be determined by well characterized assays such as reduction of carrageenan-induced paw edema in rats (Gabor, M., Mouse Ear Inflammation Models and their Pharmacological Applications, 2000).

U.S. Pat. No. 6,040,147 describes both prognostic and diagnostic applications of the measurement of levels of particular molecules including certain cytokines (e.g. interleukins 1-17) and cellular adhesion molecules (e.g. sICAM, integrins, ICAM-1, ICAM-3, BL-CAM, LFA-2, VCAM-1, NCAM and PECAM). The presence of such markers may be determined by methods well known in the art, including ELISA (enzyme linked immunosorbent assay) and other immunoassays and can be measured in body fluid, for example, blood, lymph, saliva and urine. U.S. Pat. No. 6,180,643 also describes the use of molecules such as IL-1, TNF-α as markers of IDDM and NDDM in particular, where certain therapies involve inhibiting the production of these molecules.

Chroman derivatives of the present invention are further tested in a model of muscle performance. Briefly, human subjects who are not customarily involved in weight training are given either placebo or a pre-determined daily dose of a formulation of the invention. Blood metabolites and inflammatory markers are measured prior to and at defined time intervals after eccentric exercise (for example, a defined arm “curl”) on an exercise machine. Subjective pain assessment is also taken. Antiinflammatory chroman formulations provide reduction in at least one or more markers of inflammation, as defined herein, or reduction in pain, as compared to placebo-treated control subjects.

Methods of Using Compounds of the Invention

The compositions of the present invention are administered to a mammalian subject to reduce elevated levels of the inflammatory marker, including for example CRP, associated with inflammation or to maintain and promote healthy and/or normal levels of inflammatory marker associated with inflammation. Healthy or normal ranges of such inflammatory markers are known in the art. See for example, U.S. Pat. No. 6,040,147 which provides healthy or normal ranges for CRP. For example, compositions comprising chroman derivatives of the present invention are administered to a mammalian subject at risk for developing inflammation associated with diseases or disorders disclosed herein, such as, for example, ESRD, in order to maintain healthy or normal levels of CRP. The compositions of the present invention are administered to a mammalian subject to reduce elevated levels of proteins associated with inflammation, such as for example, CRP, certain cytokines associated with inflammation as described herein.

Moreover, CRP is a significant predictor of cardiovascular and all-cause mortality in dialysis patients (Foley R N, et al., J Am Soc Nephrology 1998;9:S16-23; Handelman G J, et al., Kidney Int 2001;59:1960-6; Zimmerman J, et al., Kidney Int 1999;55:648-58; Iseki K, et al., Nephrol Dial Transplant 1999;14:1956-60).

Compounds and Compositions

Some 6-hydroxychromans described herein can be isolated as metabolites of tocopherol, see e.g. U.S. Pat. No. 6,150,402, or can be synthesized as described in for example in U.S. Pat. No. 6,048,891 or U.S. patent Application No. 2001/0031782.

The compositions, as described above, can be prepared as a medicinal preparation (such as an aqueous solution for injection) or in various other media, such as foods for humans or animals, including medical foods and dietary supplements. A “medical food” is a product that is intended for the specific dietary management of a disease or condition for which distinctive nutritional requirements exist. By way of example, but not limitation, medical foods may include vitamin and mineral formulations fed through a feeding tube to burn victims (referred to as enteral administration or gavage administration). A “dietary supplement” shall mean a product that is intended to supplement the human diet and is typically provided in the form of a pill, capsule, tablet or like formulation. By way of example, but not limitation, a dietary supplement may include one or more of the following ingredients: vitamins, minerals, herbs, botanicals, amino acids, dietary substances intended to supplement the diet by increasing total dietary intake, and concentrates, metabolites, constituents, extracts or combinations of any of the foregoing. Dietary supplements may also be incorporated into food stuffs, such as functional foods designed to promote tissue health or to prevent inflammation. If administered as a medicinal preparation, the composition can be administered, either as a prophylaxis or treatment, to a patient in any of a number of methods. The subject compositions may be administered alone or in combination with other pharmaceutical agents and can be combined with a physiologically acceptable carrier thereof. The effective amount and method of administration of the particular formulation can vary based on the individual subject, the stage of disease, and other factors evident to one skilled in the art. During the course of the treatment, the concentration of the subject compositions may be monitored to insure that the desired level is maintained.

Generally, the route(s) of administration useful in a particular application are apparent to one of skill in the art. Routes of administration include, but are not limited to, oral, topical, dermal, transdermal, transmucosal, epidermal, parenteral, gastrointestinal.

For in vitro or ex vivo administration, the compounds may be provided in the medium of the cells and/or organ, as a single bolus, by repetitive addition, by continual infusion, or the like.

For administration, the invention includes subject compositions suitable for oral administration including, but not limited to, pharmaceutically acceptable tablets, capsules, powders, solutions, dispersions, or liquids or compounded with other physiologically acceptable materials which can be ingested including, but not limited to, foods, including, but not limited to, food bars, beverages, powders, cereals, cooked foods, food additives and candies. For rectal administration, the subject compositions may be provided as suppositories, as solutions for enemas, or other convenient application. Otherwise, the subject compositions may be administered intravascularly, arterially or venous, subcutaneously, intraperitoneally, intraorganally, intramuscularly, by dermal patch, or the like.

For administration, the formulations may conveniently be presented in unit dosage form and may be prepared by any methods well known in the art of pharmacy. Such methods include the step of bringing into association the active ingredients with the carrier which constitutes one or more accessory ingredients. In general, the formulations are prepared by uniformly and intimately bringing into association the active ingredients with liquid carriers or finely divided solid carriers or both, and then if necessary shaping the product.

When the composition is incorporated into various media such as foods, it may simply be orally ingested. The food can be a dietary supplement (such as a snack or wellness dietary supplement) or, especially for animals, comprise the nutritional bulk (e.g., when incorporated into the primary animal feed).

The amount of the composition ingested, consumed or otherwise administered will depend on the desired final concentration. Typically, the amount of a single administration of the composition of the invention can be about 0.1 to about 1000 mg per kg body weight, or about 0.5 to about 10,000 mg per day. To determine the optimum concentration for any application, conventional techniques may be employed. Thus, for in vitro and ex vivo use, a variety of concentrations may be used and various assays employed to determine the degree of inflammation.

The compositions of the present invention may be administered parenterally including intravascularly, arterially or venous, subcutaneously, intradermally, intraperitoneally, intraorganally, intramuscularly, or the like.

Formulations for parenteral administration include aqueous and non-aqueous isotonic sterile injection solutions which may contain buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose sealed containers, for example, ampules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets of the kind previously described.

For topical administration, the subject compositions may be provided as a wide variety of product types including, but are not limited to, lotions, creams, gels, sticks, sprays, ointments and pastes. These product types may comprise several types of formulations including, but not limited to solutions, emulsions, gels, solids, and liposomes.

Compositions useful for topical administration of the compositions of the present invention formulated as solutions typically include a pharmaceutically-acceptable aqueous or organic solvent. The terms “pharmaceutically-acceptable organic solvent” refer to a solvent which is capable of a compound of the present invention dispersed or dissolved therein, and of possessing acceptable safety properties (e.g., irritation and sensitization characteristics). Examples of suitable organic solvents include: propylene glycol, polyethylene glycol (200-600), polypropylene glycol (425-2025), glycerol, 1,2,4-butanetriol, sorbitol esters, 1,2,6-hexanetriol, ethanol, isopropanol, butanetriol, sorbitol esters, 1,2,6-hexanetriol, ethanol, isopropanol, butanediol, and mixtures thereof.

If the topical compositions useful in the subject invention are formulated as an aerosol and applied to the skin as a spray-on, a propellant is added to a solution composition. Examples of propellants useful herein include, but are not limited to, the chlorinated, fluorinated and chloro-fluorinated lower molecular weight hydrocarbons.

Topical compositions useful in the subject invention may be formulated as a solution comprising an emollient. As used herein, “emollients” refer to materials used for the prevention or relief of dryness, as well as for the protection of the skin. A wide variety of suitable emollients is known and may be used herein.

Another type of product that may be is a cream. Another type of product that may be formulated from a subject solution is a lotion.

Yet another type of product that may be formulated from a composition of the present invention is an ointment. An ointment may comprise a simple base of animal or vegetable oils or semi-solid hydrocarbons (oleaginous). Ointments may also comprise absorption ointment bases which absorb water to form emulsions. Ointment carriers may also be water soluble.

Another type of formulation is an emulsion. Emulsifiers may be nonionic, anionic or cationic and examples of emulsifiers are described in, for example, U.S. Pat. Nos. 3,755,560, and 4,421,769.

Lotions and creams can be formulated as emulsions as well as solutions.

Single emulsions for topical preparations, such as lotions and creams, of the oil-in-water type and water-in-oil type are well-known in the art. Multiphase emulsion compositions, such as the water-in-oil-in-water type, are also known, as disclosed, for example, in U.S. Pat. No. 4,254,105. Triple emulsions are also useful for topical administration of the present invention and comprise an oil-in-water-in-silicone fluid emulsion as disclosed, for example in U.S. Pat. No. 4,960,764.

Another emulsion useful in the topical compositions is a micro-emulsion system. For example, such a system comprises from about 9% to about 15% squalane, from about 25% to about 40% silicone oil; from about 8% to about 20% of a fatty alcohol; from about 15% to about 30% of polyoxyethylene sorbitan mono-fatty acid (commercially available under the trade name TWEENS) or other nonionics; and from about 7% to about 20% water.

Liposomal formulations are also useful for the compositions of the present invention. Such compositions can be prepared by combining a compound of the present invention and/or mixtures thereof, with a phospholipid, such as dipalmitoylphosphatidyl choline, cholesterol and water according to known methods, for example, as described in Mezei et al. (1982) J. Pharm. Pharmacol. 34:473-474, or a modification thereof. Epidermal lipids of suitable composition for forming liposomes may be substituted for the phospholipid. The liposome preparation is then incorporated into one of the above topical formulations (for example, a gel or an oil-in-water emulsion) in order to produce the liposomal formulation. Other compositions and pharmaceutical uses of topically applied liposomes are described for, example, in Mezei (1985) Topics in Pharmaceutical Sciences, Breimer et al. eds., Elsevier Science, New York, N.Y., pp. 345-358.

For rectal administration, the subject compositions may be provided as solutions for enemas, as suppositories with a suitable base comprising, for example, cocoa butter or a salicylate, or as other convenient applications.

Formulation for vaginal administration may be presented as pessaries, tampons, creams, gels, pastes, foams or spray formulations containing in addition to the active ingredient such carriers as are known in the art to be appropriate.

To determine the optimum concentration for any application, conventional techniques may be employed. Thus, for in vitro and ex vivo use, a variety of concentrations may be used and various assays employed to determine the degree of dysfunction of the cells when exposed to stress. Examples of such assays are described herein and have been described, for example, in U.S. Pat. No. 5,801,159.

The above-mentioned compositions and methods of administration are meant to describe but not limit the methods and compositions of the present invention. The methods of producing various compositions and devices are within the ability of one skilled in the art and are not described in detail here.

The compositions comprising chroman including the compositions comprising tocopherol metabolites of the present invention are capable of reducing the level of inflammatory markers associated with inflammation, such as for example, CRP and IL-6. These conditions can be induced experimentally by chemical interference or by changing the environmental conditions in the laboratory (e.g., by inducing anoxia, hypothermia, hyperthermia, etc.).

Various assays, compositions and methods useful for identifying compositions and methods for reducing tissue damage are provided in the Examples.

The following examples are provided to illustrate, but not limit, the invention.

### EXAMPLES

### Example 1

### Cellular Inflammation

This example provides exemplary assays for measuring inflammatory reaction in a cell line. Specifically, this assay provides a predictive measure of anti-inflammatory activity of formulations of the present invention.

A. Human Hep3B Cells—CRP assay. Hep3B Cell Line is obtained from the American Type Culture Collection (ATCC Catalog No. HB-8064). The Hep3B cell line was derived from liver tissue of an 8-year-old Black male. The cells are epithelial in morphology and produce tumors in nude mice. The cells produce α-fetoprotein, hepatitis B surface antigen, albumin, α-2-macroglobulin, α-1-antitrypsin, transferrin, plasminogen, complement C3 and β-lipoprotein (Knowles B B, et al., Science, 1980, 209:497499). This cell line has been widely used to study hepatocyte cytokine and acute phase protein release (e.g., Damtew B, et al., 1993, J. Immunol. 150:4001-4007).

HEP3B cells are grown in Minimum Essential Medium (MEM; GIBCO) supplemented with 10% Fetal Bovine Serum (FBS; Hyclone), 1× Penicillin/Streptomycin (GIBCO, Cat#. 15140-122) and 0.1 mM non-essential amino acids (GIBCO, Catalog No. 11140-050). Cells are thawed and transferred to warm medium according to standard methods known in the art.

Cells are incubated in flasks at 37° C. with 5% CO₂ in an air atmosphere incubator. HEP3B growth media is changed every 2 days until the cells reach 70-80% confluence (approx. 3-4 days). For assay, the cells are transferred to 96-well plates, seeded at 5000 cells per well in culture media, and left to grow for 7 days in a 37° C. incubator (air supplemented with 5% CO₂). Media is replaced daily until assay.

Test compounds are diluted into “Stimulus Buffer” (MEM medium containing 0.1 mM non-essential amino acids, 1× penicillin/streptomycin, 10% FBS with 10 ng/ml IL-1β, 20 ng/ml IL-6 and 1 μM dexamethasone. Media is removed from the cells and is replaced with 200 μl of test dilution. Cells are returned to the incubator for three days at 37° C. CRP ELISA is then performed on supernatant from the cells, as described below.

Costar EIA/RIA plates are coated with rabbit anti-human CRP (DAKO) diluted 1:4000 in carbonate buffer (100 μl/well) for 45 minutes at 37° C. Plates are then washed 5× with CRP washing buffer (50 mM Tris-HCl, 0.3M NaCl, 0.5 MI Tween-20, pH 8.0) using an automatic plate washer. Plates may be dried, covered and refrigerated until use. Supernatant (100 μl) is removed from each well of the test plates and added to the corresponding well of a precoated ELISA plate.

100 μl HRP-conjugated rabbit anti-human CRP (DAKO) diluted 1:500 (in CRP wash buffer) is added to each well, followed by incubation for 30 minutes at 37° C. Plates are washed 5× with CRP washing buffer using the automatic plate washer. 200 μl of 3,3′,5,5′-Tetramethyl Benzidine (TMB) liquid Substrate System (Sigma, St. Louis, Mo.) is added to each well, followed by incubation in the dark for 15 minutes at room temperature. Finally, 50 μl of 1M H₂SO₄ is added to each well and absorbance at 450 nm is immediately measured in a microtiter spectrophotometer.

CRP measured as above is normalized to cell count per well, using a cell viability assay, such as the Cell Tracker Green assay. To do this, the remainder of the medium is from the cell test plates, cells are washed with 200 μL of pre-warmed 1× Hanks Basic Salt Solution (HBSS; GIBCO), and 100 μL of 5 μM Cell Tracker Green (Molecular Probes, Eugene, Oreg.) is added to each well. Plates are then incubated at 37° C. for 30 minutes. Cells are then washed twice with prewarmed 1×HBSS. Plates are immediately read using a Fluoroskan® flourometer with a 485 excitation/538 emission filter pair.

In a CRP assay such as the one disclosed herein, 3-(6-hydroxy-2,7,8-trimethyl-chroman-2-yl)propionic acid methyl ester at an EC₅₀ of between about 10 to about 30 μM and 3-(6-hydroxy-2,7,8-trimethyl-chroman-2-yl)-propionic acid at an EC₅₀ of between about 40 to about 60 μM was effective at reducing CRP levels. Other chromans can be tested in this assay to determine their reduction of CRP levels.

B. Cell-ELAM Assay. Endothelial-Leukocyte Adhesion Molecule (ELAM), also known as E-selectin, is expressed on the surface of endothelial cells. In this assay, lipopolysaccharide (LPS) and IL-1β are used to stimulate the expression of ELAM; test agents are tested for their abilities to reduce this expression, in accordance with studies showing that reduction of leukocyte adhesion to endothelial cell surface is associated with decreased cellular damage (e.g., Takada, M., Et al., Transplantation 64: 1520-25, 1997; Steinberg, J. B., et al., J. Heart Lung Trans. 13:306-313, 1994).

Endothelial cells may be selected from any of a number of sources and cultured according to methods known in the art; including, for example, coronary artery endothelial cells, human brain microvascular endothelial cells (HBMEC; Hess, D. C., et al., Neurosci. Lett. 213(1): 37-40,1996), or lung endothelial cells. Cells are conveniently cultured in 96-well plates. Cells are stimulated by adding a solution to each well containing 10 μg/ml LPS and 100 μg/ml IL-10 for 6 hours in the presence of test agent (specific concentrations and time may be adjusted depending on the cell type). Treatment buffer is removed and replaced with pre-warmed Fixing Solution® (100 μl/well) for 25 minutes at room temperature. Cells are then washed 3×, then incubated with Blocking Buffer (PBS+2% FBS) for 25 minutes at room temperature. Blocking Buffer containing Monoclonal E-Selectin Antibody (1:750, Sigma Catalog #S-9555) is added to each well. Plates are sealed and stored at 4° overnight. Plates are washed 4× with 160 μL Blocking Buffer per well. Second Antibody-HRP diluted 1:5000 in Blocking Buffer is then added (100 μL/well), and plates are incubated at room temperature (protected from light) for two hours. Plates are then washed 4× with Blocking Buffer before addition of 100 μL of ABTS Substrate solution at room temperature (Zymed, Catalog #00-2024). Wells are allowed to develop for 35 minutes, before measurement at 402 nm in a Fluoroskan® Reader with shake program for 10 seconds. Positive results are recorded as a decrease in ELAM concentration in tested wells, as compared to control wells.

In an ELAM assay, such as the one described herein, the chromans of the present invention such as:

3-(5-Bromo-6-hydroxy-2,7,8-trimethyl-chroman-2-yl)-propionic acid methyl ester;

3-(7,8-Dihydroxy-2-methyl-chroman-2-yl)-propionic acid; and

6-Hydroxy-2,5,7,8-tetramethyl-chroman-2-carboxylic acid (adamantan-2-ylmethyl)-amide;

at EC₅₀ in a range of 10-100 μM, and in particular in a range of 20-60 μM, were able to reduce the expression of ELAM.

### Example 2

### Determination of Activity Utilizing Neuronal Cell Stress Assay

A. Isolation and Culture of Primary Hippocampal Neuronal Cells.

Materials

Neurobasal/B27i: Neurobasal medium (Life Technologies, Rockville, Md.) with 1×B27 supplement (Life Technologies), 0.5 μM L-glutamine, 25 μM L-glutamic acid, and 1× Penicillin/Streptomycin.

Hank's Basic Salt Solution (HBSS, Ca/Mg-free) was prepared by preparing 1×Hanks CMF (Gibco) supplemented with HEPES (10 mM, pH 7.3), sodium bicarbonate (0.35%), 1× Penicillin/Streptomycin, and 1 mM pyruvate.

Poly-D-lysine (Sigma, St. Louis, Mo.), 50 μg/ml solution filtered through 0.2 μm filter tubes.

Sigmacote (Sigma, St. Louis, Mo.).

Plastic Culture Flasks (T75 cm²) or 12-well cell culture plates treated with Poly-D-Lysine (Sigma, St. Louis, Mo.).

Preparation of Primary Hippocampal Neuronal Cells

A pregnant female mouse (E18-E19) was euthanized with CO₂ prior to removal of the uterus, which was then placed in a sterile plastic petri dish. The embryos were removed from the sac, and the embryonic brains were removed and immersed in cold (4° C.) Buffered Salt Solution (HBSS; Ca/Mg free; Life Technologies) in a small petri dish. Hippocampi were then removed from the brains under a dissecting microscope and were placed on a paraffin-covered dish. The meninges were stripped away and the dissected hippocampi were collected in a small petri dish in HBSS. The hippocampi were transferred to a 15-ml centrifuge tube (normally 10-12 brains) filled with HBSS. The tube containing the brains was centrifuged at 1000 rpm for 2 min in a tabletop centrifuge. The supernatant was removed, 2 ml of HBSS was added to the hippocampi in the tube, and the resulting suspension was triturated 2 times each with long-tipped siliconized glass pipettes having progressively smaller apertures, starting with a pipette with a standard size opening (approximately 1.0 mm diameter), following with one having an aperture of half standard size (approximately 0.5 mm diameter), then with one having an aperture about one-half that size (0.25 mm diameter). The suspension was then centrifuged again at 1000 rpm for 2 min in a tabletop centrifuge, the supernatant was discarded, and 2 ml of Neurobasa/B27i (with antibiotics) was added to the tube. The trituration procedure described above was then repeated on this suspension.

The density of cells was determined on a small aliquot of cells using standard counting procedures and correcting for cell viability by trypan blue stain exclusion. Using this procedure, the expected yield is 3×10⁵-6×10⁵ cells/brain. Cells were then added to PDL-coated 12 well plates, flasks or MetTek dishes in Neurobasal/B27l at a density of about 1.5×10⁶ cells (T75 flask) or about 100,000 cells/well of a 12-well plate. Plated cells were incubated at 37 degrees in an atmosphere of 5% CO₂/95% O₂. Media was renewed after 3-4 days by replacing half of it with fresh Neurobasa./B27m medium, containing 5 □M cytosine arabinoside (AraC). Seven to eight days from the initial culture, the media was renewed again, by removing one-half or it and replacing with an equal amount of fresh Neurobasal/B27m medium (without Ara-C).

B. Hippocampal Anoxia-Reoxygenation Cell Death Assay.

This assay was used to induce ischemia by anoxia-reoxygenation in cultured hippocampal neuronal cells. Test compounds were added to assess potency and efficacy against ischemia-induced neuronal cell injury and cell death.

Materials.

Neurobasal media, NoG neurobasal media, B27 supplement and B27 Supplement minus AO were obtained from Life Technologies.

Neurobasal/B27 medium was prepared with 2×B27 minus AO supplement, 0.5 mM L-glutamine and 0.25× penicillin/streptomycin. Cell Tracker Green was obtained from Molecular Probes and a fresh 5 μM solution was prepared from 10 mM stock just before use.

NoG-Neurobasal contains NoG neurobasal medium plus 0.5 mM glucose, 0.1 mM L-glutamine and 0.25× Penicillin/Streptomycin.

Primary hippocampal neuronal cells were prepared according to the methods described above and were cultured in poly-D-lysine coated 12 well plates for 10-11 days prior to use.

Deoxygenated LoG-Neurobasal medium (100 ml) was prepared by pre-equilibrating the medium in a T150 cm² flask in a hypoxic chamber overnight. Following pre-incubation under hypoxic conditions, the LoG-Neurobasal media was lightly bubbled with 100% N₂ for 30 min to completely deoxygenate the media. An additional 20 ml LoG-Neurobasal were pre-equilibrated in a T75 cm² flask and 100 ml Neurobasal/B27AO was incubated in a normal incubator (5% CO₂) overnight. Reoxygenated medium was prepared by placing medium overnight in the culture incubator (5% CO₂/95% O₂) prior to use.

Existing culture medium (Neurobasal/B27m) was removed from the cells by aspiration. Cells were washed once with 2 ml/well (12-well culture plates) of glucose free-BSS. Neurons were replenished 10-11 days after initial culture with deoxygenated LoG-Neurobasal (1 ml per well for each well of a 12-well plate). Test compounds were added directly to each well (usually 3 concentrations of the compound plus positive control, each in triplicate). Most test compounds were dissolved in 100% DMSO; however, concentrations were adjusted such that the final concentration of DMSO in the cell media never exceeded 0.5%. Plates containing cells with test compounds were placed in a hypoxic chamber for 5 hr with plate lids ajar. For normoxia controls, pre-equilibrated normoxic LoG-Neurobasal medium was added to each well of cells, and the plate was replaced in the normal culture incubator for 5 hr. After 5 hr of hypoxia, the existing media was carefully aspirated off, and 2 mL of new, reoxygenated (pre-equilibrated) Neurobasal/B27AO was added to each well. The same test compounds (in the same the concentrations) were added back into the corresponding wells. Plates were placed in the cell culture incubator (5% C₂/95% O₂) and reoxygenated for 20-24 hr. After reoxygenation for 20-24 hr, live neurons were quantitated using the cell tracker green fluorescence method, described below.

To test for cell viability, existing culture medium was aspirated from each well of the 12 well plates, and neurons were washed once with 2 ml of HBSS (pH 7.4, prewarmed to 30-37° C.). To each well was added one milliliter of 5 μM Cell Tracker Green fluorescent dye dissolved in HBSS. Plates were placed in the dark at room temperature for 15 minutes, and then were washed with two milliliters of HBSS. One milliliter of HBSS was then added to each well, and fluorescent cells were counted using a fluorescent microscope. Significantly increased cell viability compared to control cells is indicative of a protective compound.

Compounds of the present invention were tested as described above and provided protection against stressor-induced cell death in at least about 20% of the cells tested, at concentrations ranging from about 1 to 100 μM.

### Example 3

### In Vivo Model of Cellular Inflammation

This assay measures the ability of test compounds to prevent or reduce inflammation secondary to oxazolone or arachidonic acid.

A. Arachidonic acid. Albino male CD-1 mice, 7-9 weeks old were used in this test. A 20% (w/v) arachidonic acid solution in acetone was prepared. Twenty microliters of the arachidonic acid solution was applied to the dorsal left ear of the mouse. Immediately thereafter, test compounds (20 μL in 70% ethanol/30% propylene glycol) were applied to the left ear. The untreated right ears served as control. Mice are sacrificed by CO₂ inhalation, one hour after treatment. The left and right ears were removed and 7 mm punch biopsies taken from each. The punch biopsies were weighed, and the differences calculated.

Certain compounds of the present invention were tested as described above.

3-(7,8-Dihydroxy-2-methyl-chroman-2-yl)-propionic acid, and

3-(5-Bromo-6-hydroxy-2,7,8-trimethyl-chroman-2-yl)-propionic acid methyl ester showed about 40% protection in this assay.

B. Oxazolone. CD-1 mice are induced by applying 3% oxazolone (Sigma) (30 mg/ml prepared in corn oil:acetone) to the shaved abdomen. Five days later, the mice are challenged with 2% oxazolone (20 mg/ml) in acetone on the left ear (right ear was untreated control). One hour after challenge, test compounds are applied to the left ear in 70% ethanol/30% propylene glycol. Animals are sacrificed 24 hours later and 7 mm ear punches are removed. The ear punches are placed on a balance scale, and the difference between the untreated and treated ears is determined. Percent inhibition is calculated by comparing the means of each group to the vehicle group. (Hydrocortisone serves as a positive control in this test.). compounds of the present invention can be tested for their ability to reduce inflammation in this model.

### Example 4

### Muscle Inflammation

Healthy, non-exercising young male adults (aged 18-25) provide a sample of blood for baseline metabolite testing (defined below) and are given test article or placebo for seven days. Subjects then perform three sets of ten repetitions using 80% of their eccentric 1-repetition maximum on a Cybex® arm curl machine (Cybex International Inc., Medway, Mass.). The subject is given two minutes rest between sets, and repetitions continue until fatigue. Three days following exercise, subjects provide blood for testing of CBC and levels of metabolites and markers (isoprostanes, lipid hydroperoxides, LDH, CK, myoglobin, CRP, IL-6, myeloperoxidase). Subjects also provide a subjective evaluation of muscle soreness. These indices are measured and collected again four days later.

Baseline levels of metabolites and markers are taken as the mean of values measured at intake and just prior to exercise; differences from baseline are calculated 3 and 7 days post exercise. Test compounds are considered to have an anti-inflammatory effect, if they produce a decrease in any of the inflammatory markers, in particular, CRP, compared to baseline or if they produce a reduction in the average increase measured in control (placebo-treated) subjects. Chromans of the present invention can be tested for their ability to reduce inflammatory markers, such as for example, CRP levels in this model.

### Example 5

### Carrageenan-Induced Rat Paw Edema Assay

Methods

Animal Preparation: Male Sprague-Dawley rats weighing between 175 to 200 g are used in this study. Animals are allowed free access to water and commercial rodent diet under standard laboratory conditions. Room temperature is maintained at 20-23° C. and room illumination is on a 12/12-hour light/dark cycle. Animals are acclimatized to the laboratory environment 5 to 7 days prior to the study.

Experimental Procedure

Each animal is treated by administration of vehicle, reference or test substance one hour prior to carrageenan injection, as follows:

I.V. Infusion via Femoral Vein: Anesthesia is maintained by inhalation of 3.0% isoflurane (Aerrane, Front Dodge, Iowa) in oxygen throughout the entire procedure. The exterior site of the right femoral vein is shaved and sterilized prior to surgery. A 3-cm incision is made in the right groin region and the femoral vein is isolated. The femoral vein is temporarily ligated with a micro-vascular clip, and a small incision is made on the femoral vein to introduce and advance a polyethylene (PE-50) catheter (Becton. Dickinson and Co., Sparks, Md.). The catheter is secured in place with suture (silk 5/0, Carlisle Laboratories, Farmers Branch, Tex.). The other end of the catheter is attached to a syringe filled with the saline for the bolus injection. Using a hemostat, a pocket is made subcutaneously on the back of the animal so the PE catheter could be brought up to the exteriorization point between the shoulder blade for either a bolus injection or a continuous injection by an osmotic pump.

Gavage Feeding: A standard rat gavage tube (Popper & Sons Inc, NY) is attached to a 3-cc hypodermic syringe. The animal is held in a vertical position. The feeding tube is placed into the mouth and then gently advanced until it reached the stomach. The content of the syringe is slowly delivered, and then the tube is withdrawn.

I.P. Injection: An awake rat is held in a standard hand held position. A 23 ¾ G needle is injected into the lower right quarter of the abdomen pass the peritoneum, slightly off the midline. To avoid organ injection, the plunger of the syringe is slightly pulled back. If no fluid is withdrawn, the content of the syringe is delivered into the abdominal cavity.

One hour post treatment each animal is anesthetized with 3.0% isoflurane (Aerrane, Front Dodge, Iowa) in oxygen and administered 100 μl of 1% Carrageenan Lambda type IV (Sigma Chemical Company, St. Louis, Mo.) suspension (freshly made, within 2-3 hours of use) in saline, into the intraplantar surface of the right hind paw. Paw edema is measured four hours after carrageenan injection, either by measuring the increase in paw volume using a plethysmometer or the increase in paw weight using a fine scale. Immediately prior to edema measurement, the animals are euthanized via CO₂ asphyxiation and 500 μl blood is withdrawn by cardiac puncture for later analysis. Paw volume is determined by the extent to which water is displaced by the paw from a pre-calibrated chamber. The volume of the left hind paw (control) is subtracted from the volume of the right hind paw (carrageenan-treated) to determine the volume of carrageenan-induced edema. To measure the weight difference between paws, both hind paws are removed and weighed separately. Edematous fluid is removed and tested for inflammatory markers, such as IL-6, using standard ELISA techniques known in the art.

Statistical Analysis

The difference of the weight or the volume or biomarker level between right and left paw is calculated for each animal for the analysis. Group data are presented as means+/−SEM, and p<0.05 is considered significant. Inter-group comparisons are carried out by unpaired student t test (between two groups) or one-way ANOVA followed by post hoc Bonferroni's multiple comparisons.

It is claimed:

1. A method of reducing the level of C-reactive protein (CRP) in an individual subject to a CRP associated inflammatory condition, comprising administering to the individual an effective amount of a composition comprising a compound of Formula I: 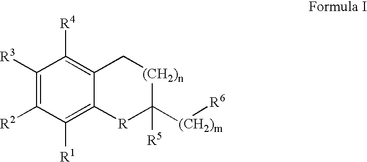
wherein:
R is O, S, SO, SO₂, a secondary or tertiary amine, a phosphate, a phosphoester, or a substituted or unsubstituted methylene group;
R¹ and R² independently are H, OH, alkyl, aryl, alkenyl, alkynyl, ether, ester, amine, amide, halogen, or sulfonyl, or jointly complete a 5- or 6-membered aliphatic or aromatic ring;
R³ and R⁴ independently are H, OH, alkyl, aryl, alkenyl, alkynyl, ether, ester, amine, amide, nitro, halogen, or sulfonyl, or jointly complete a 5- or 6-membered aliphatic, aromatic or heterocyclic ring;
R⁵ is H, OH, alkyl, aryl, alkenyl, alkynyl, ester, or amine;
R⁶ is COOH, COOR⁷, CONH₂, CONHR⁷, CONR⁷R⁸, NH₂, NHR⁷, NR⁷R⁸, OH, or OR⁹;
R⁷ and R⁸ independently are alkyl, aryl, aralkyl, alkenyl, or alkynyl;
R⁹ is alkyl, aralkyl, alkenyl, alkynyl, or a glucoside;
n is 0 to 3; and
m is 0 to 5;
or individual isomer, racemic or non racemic mixture of isomers, of pharmaceutically acceptable salt or solvate thereof.

2. The method of claim 1, wherein the compound is selected from the group:
6-Hydroxy-2,5,7,8-tetramethyl-chroman-2-carboxylic acid;
6-Hydroxy-2,5,7,8-tetramethyl-chroman-2-carboxylic acid (adamantan-2-ylmethyl)-amide;
2-Hydroxymethyl-6-(6-hydroxy-2,5,7,8-tetramethyl-chroman-2-ylmethoxy)-tetrahydro-pyran-3,4,5-triol; 3-(6-Hydroxy-2-methyl-chroman-2-yl)-propionic acid methyl ester;
3-(6-Hydroxy-2,7,8-trimethyl-chroman-2-yl)-propionic acid methyl ester;
3-(6-Hydroxy-2,7,8-trimethyl-chroman-2-yl)-propionic acid;
3-[8-(2-Methoxycarbonyl-ethyl)-3,5,6,8-tetramethyl-1,2,3,8,9,10-hexahydro-pyrano[3,2-f]chromen-3-yl]-propionic acid methyl ester;
3-[8-(2-Carboxy-ethyl)-3,5,6,8-tetramethyl-1,2,3,8,9,10-hexahydro-pyrano[3,2-f]chromen-3-yl]propionic acid;
3-(6-Hydroxy-2-methyl-chroman-2-yl)-propionic acid;
3-(6-Hydroxy-2,5,7,8-tetramethyl-chroman-2-yl)-propionic acid;
3-(2,5,7,8-Tetramethyl-chroman-2-yl)-propionic acid;
3-(6-Hydroxy-2,7,8-trimethyl-5-nitro-chroman-2-yl)-propionic acid;
3-(6-Hydroxy-2-methyl-3,4-dihydro-2H-benzo[h]chromen-2-yl)-propionic acid;
3-(5-Bromo-6-hydroxy-2,7,8-trimethyl-chroman-2-yl)-propionic acid methyl ester;
3-(5-Bromo-6-hydroxy-2,7,8-trimethyl-chroman-2-yl)-propionic acid;
3-(7,8-Dihydroxy-2-methyl-chroman-2-yl)-propionic acid; and
6-Hydroxy-2,5,7,8-tetramethyl-chroman-2-carboxylic acid.

3. The method of claim 1, wherein the compound is selected from the group:
6-Hydroxy-2,5,7,8-tetramethyl-chroman-2-carboxylic acid (adamantan-2-ylmethyl)-amide;
2-Hydroxymethyl-6-(6-hydroxy-2,5,7,8-tetramethyl-chroman-2-ylmethoxy)-tetrahydro-pyran-3,4,5-triol;
3-(6-Hydroxy-2-methyl-chroman-2-yl)-propionic acid methyl ester;
3-[8-(2-Methoxycarbonyl-ethyl)-3,5,6,8-tetramethyl-1,2,3,8,9,10-hexahydro-pyrano[3,2-f]chromen-3-yl]-propionic acid methyl ester;
3-[8-(2-Carboxy-ethyl)-3,5,6,8-tetramethyl-1,2,3,8,9,10-hexahydro-pyrano[3,2-f]chromen-3-yl]propionic acid;
3-(6-Hydroxy-2-methyl-chroman-2-yl)-propionic acid;
3-(2,5,7,8-Tetramethyl-chroman-2-yl)-propionic acid;
3-(6-Hydroxy-2,7,8-trimethyl-5-nitro-chroman-2-yl)-propionic acid;
3-(6-Hydroxy-2-methyl-3,4-dihydro-2H-benzo[h]chromen-2-yl)-propionic acid;
3-(5-Bromo-6-hydroxy-2,7,8-trimethyl-chroman-2-yl)-propionic acid methyl ester;
3-(5-Bromo-6-hydroxy-2,7,8-trimethyl-chroman-2-yl)-propionic acid; and
3-(7,8-Dihydroxy-2-methyl-chroman-2-yl)-propionic acid.

4. The method of claim 1, wherein the compound is selected from 3-(6-hydroxy-2,7,8-trimethylchroman-2-yl)-propionic acid and 3-(6-hydroxy-2,7,8-trimethyl-chroman-2-yl)-propionic acid methyl ester.

5. The method of claim 1, wherein the compound is selected from 3-(5-bromo-6-hydroxy-2,7,8-trimethyl-chroman-2-yl)-propionic acid methyl ester and 3-(5-bromo-6-hydroxy-2,7,8-trimethyl-chroman-2-yl)-propionic acid.

6. A method of reducing the level of an inflammatory marker in an individual subject to end-stage renal disease comprising administering to the individual a composition comprising a compound of claim 1 in an effective amount.

7. The method of claim 6, wherein said inflammatory marker is C-reactive protein (CRP).

8. The method of claim 6, wherein said composition comprises a compound selected from the group:
6-Hydroxy-2,5,7,8-tetramethyl-chroman-2-carboxylic acid;
6-Hydroxy-2,5,7,8-tetramethyl-chroman-2-carboxylic acid (adamantan-2-ylmethyl)-amide;
2-Hydroxymethyl-6-(6-hydroxy-2,5,7,8-tetramethyl-chroman-2-ylmethoxy)-tetrahydro-pyran-3,4,5-triol;
3-(6-Hydroxy-2-methyl-chroman-2-yl)-propionic acid methyl ester;
3-(6-Hydroxy-2,7,8-trimethyl-chroman-2-yl)-propionic acid methyl ester;
3-(6-Hydroxy-2,7,8-trimethyl-chroman-2-yl)-propionic acid;
3-[8-(2-Methoxycarbonyl-ethyl)-3,5,6,8-tetramethyl-1,2,3,8,9,10-hexahydro-pyrano[3,2-f]chromen-3-yl]-propionic acid methyl ester;
3-[8-(2-Carboxy-ethyl)-3,5,6,8-tetramethyl-1,2,3,8,9,10-hexahydro-pyrano[3,2-f]chromen-3-yl]-propionic acid;
3-(6-Hydroxy-2-methyl-chroman-2-yl)-propionic acid;
3-(6-Hydroxy-2,5,7,8-tetramethyl-chroman-2-yl)-propionic acid;
3-(2,5,7,8-Tetramethyl-chroman-2-yl)-propionic acid;
3-(6-Hydroxy-2,7,8-trimethyl-5-nitro-chroman-2-yl)-propionic acid;
3-(6-Hydroxy-2-methyl-3,4-dihydro-2H-benzo[h]chromen-2-yl)-propionic acid;
3-(5-Bromo-6-hydroxy-2,7,8-trimethyl-chroman-2-yl)-propionic acid methyl ester;
3-(7,8-Dihydroxy-2-methyl-chroman-2-yl)-propionic acid; and
6-Hydroxy-2,5,7,8-tetramethyl-chroman-2-carboxylic acid.

9. The method of claim 6, wherein the compound is selected from 3-(6-hydroxy-2,7,8-trimethylchroman-2-yl)-propionic acid and 3-(6-hydroxy-2,7,8-trimethyl-chroman-2-yl)-propionic acid methyl ester.

10. The method of claim 6, wherein the compound is selected from 3-(5-bromo-6-hydroxy-2,7,8-trimethyl-chroman-2-yl)-propionic acid methyl ester and 3-(5-bromo-6-hydroxy-2,7,8-trimethyl-chroman-2-yl)-propionic acid.

11. A method for ameliorating a symptom of an inflammatory condition in an individual subject to an inflammatory condition comprising administering to the individual a composition comprising a compound of claim 1, in an amount effective to reduce the level of an inflammatory marker associated with said inflammatory condition.

12. The method of claim 11, wherein said inflammatory marker is C-reactive protein (CRP).

13. The method of claim 11, wherein said inflammatory condition is selected from the group consisting of cardiovascular inflammatory condition, respiratory inflammatory condition, sepsis, diabetes, muscle fatigue, systemic lupus erythematosis (SLE), end stage renal disease (ESRD), premenstrual syndrome (PMS), and periodontal disease.

14. The method of claim 11, comprising administering to the individual a composition comprising 3-(6-hydroxy-2,7,8-trimethyl-chroman-2-yl)-propionic acid methyl ester in an amount effective to reduce the level of an inflammatory marker associated with said inflammatory condition.

15. The method of claim 14, wherein said inflammatory marker is C-reactive protein (CRP).

16. The method of claim 14 wherein said inflammatory condition is selected from the group consisting of cardiovascular inflammatory condition, respiratory inflammatory condition, sepsis, diabetes, muscle fatigue, SLE, renal inflammation including ESRD, premenstrual syndrome (PMS), and periodontal disease.

17. The method of claim 11, comprising administering to the individual a composition comprising 3-(5-bromo-6-hydroxy-2,7,8-trimethyl-chroman-2-yl)-propionic acid methyl ester in an amount effective to reduce the level of an inflammatory marker associated with said inflammatory condition.

18. The method of claim 17, wherein said inflammatory marker is C-reactive protein (CRP) or IL-6.

19. The method of claim 17, wherein said inflammatory condition is selected from the group consisting of cardiovascular inflammatory condition, respiratory inflammatory condition, sepsis, diabetes, muscle fatigue, SLE, renal inflammation including ESRD, premenstrual syndrome (PMS), and periodontal disease.

20. The method of claim 1, wherein said composition further comprises a pharmaceutically acceptable carrier.

21. The method of claim 6, wherein said composition further comprises a pharmaceutically acceptable carrier.

22. The method of claim 11, wherein said composition further comprises a pharmaceutically acceptable carrier.
